(19) United States
(12) Patent Application Publication
(10) Pub. No.: US 2003/0139335 A1
(43) Pub. Date: Jul. 24, 2003

(54) Enhancing organ maturity in neonates and predicting their duration of intensive care

(76) Inventors: Hartmut M. Hanauske-Abel, Edge Water, NJ (US); Axel-Rainer Hanauske, Hamburg (DE)

Correspondence Address: Michael L. Goldman, NIXON PEABODY LLP, Clinton Square, P.O. Box 31051, Rochester, NY, 14603-1051, US

(21) Appl. No.: 10/207,623

(22) Filed: Jul. 26, 2002

Related U.S. Application Data
(60) Provisional application No. 60/308,143, filed on Jul. 27, 2001.

(51) Int. Cl. A61K 48/00; A61K 38/18; C12Q 1/68; G01N 33/53

(52) U.S. Cl. 514/12; 514/44; 435/6; 435/7.1

(57) ABSTRACT

The present invention is directed to methods of enhancing organ maturity of premature human neonates or increasing collagen type IV formation in a living system by administering a growth factor, a gene encoding a growth factor, or an agent that increases growth factor formation to the living system under conditions effective to increase collagen type IV formation. Another aspect of the present invention relates to a method of predicting a premature human neonate's length of stay in a neonatal intensive care unit as well as the anticipated medical costs incurred during said stay by providing a sample from the premature human neonate and determining biomarkers derived from the extracellular matrix in the sample. The biomarker levels or ratios thereof in the sample are compared to a standard to ascertain the premature human neonate's length of stay in a neonatal intensive care unit as well as the anticipated medical costs incurred during said stay.

The present application claims benefit of U.S. Provisional Patent Application Serial No. 60/308,143, filed Jul. 27, 2001.

The invention of the present application was made with funding under National Institutes of Health Grant Nos. HD00072 and RR06020. The U.S. Government may have certain rights.

### FIELD OF THE INVENTION

The present invention is directed to enhancing organ maturity of premature human neonates and to predicting a premature human neonate's length of stay in a neonatal intensive care unit as well as the anticipated medical costs incurred during that stay. The present invention is also directed to increasing collagen type IV formation in a living system.

### BACKGROUND OF THE INVENTION

Premature birth represents a major medical challenge, and for decades has been the focus of research efforts in obstetrics and in pediatrics. Prematurity is also a major societal challenge due to its remarkable economic costs. Infants born prematurely (<37 weeks gestational age) account for only 9% of all live births, yet consume 57% of all acute neonatal intensive care unit (NICU) costs in the United States, or $ 5.8 billion annually (St. John et al., “Costs Of Neonatal Care According To Gestational Age At Birth And Survival Status,” Am. J. Obstet. Gynecol., 182:170-175 (2000)). This amount accounts for over 40% of total health care costs for all infants. Over the longer term from birth to age 15, follow-up costs due to the medical sequelae of prematurity exceed an additional $5.4 billion per year, or 10% of the health care costs for all children (Zupancic et al., “Economics Of Prematurity In The Era Of Managed Care,” Clin. Perinatol., 27(2):483-497 (2000)). The overall societal expenditures on premature birth therefore are in excess of $11 billion annually. This number does not, of course, include the direct and indirect loss in economic productivity due the long-term physical and mental handicaps experienced by survivors of prematurity and the required allocation of time and resources for their care, nor does it account for the emotional pain and anguish experienced by parents of ‘preemies’. Of note, there is general agreement that the incidence of prematurity in the United States is increasing.

The medical care for prematurely born infants is dictated by the immaturity of their bodies in general, and in particular by the immaturity of their cardiopulmonary system. Once separated from the placenta, the latter has to function competently, extracting molecular oxygen from the inhaled gases of the atmosphere and distributing the hemoglobin-bound molecular oxygen via the blood stream to all cells of the body. Besides the merely supportive interventions, such as maintaining sugar, gases, and acid/base homeostasis in the blood at the age-appropriate range, or supplying exogenous surfactant to overcome the lack of endogenous surfactant, there are also interventions that intend to accelerate tissue maturation by enhancing the activity of genes in the baby's body. The vast majority of these agents directly addressing the deficient gene expression in tissues of premature babies, are corticosteroids. Given immediately before delivery of a preemie to the mother, and modulated by the presence of her placenta, corticosteroids remarkably advance the performance level of the preemie's cardiopulmonary system after delivery.

Corticosteroids are also administered to neonates after delivery, with the expectation that postnatal administration has the same beneficial effect on the cardiopulmonary system as antenatal administration. On average, one out of four preemies in the United States and Canada is medicated with corticosteroids in the NICU, and this fraction exceeds one out of three if the birth weight is below 1000 grams. It was therefore a stunning development for those knowledgeable in the field when, in early 2002, the American Academy of Pediatrics and the Canadian Pediatric Society recommended in a joint statement that postnatal corticosteroids should no longer be used routinely in preterm infants due to the unacceptable number of short- and long-term complications (American Academy of Pediatrics and Canadian Pediatric Society, “Postnatal Corticosteroids to Treat or Prevent Chronic Lung Disease in Preterm Infants,” Pediatrics, 109:330-338 (2002)). Thus, at the time of this writing, neonatal intensive care has nothing to offer a premature baby other than sophisticated supportive therapy relying on ‘sugar water, salt, and air’.

Accordingly, there is an acute need to introduce a drug that has the potential to accelerate the extrauterine maturation of the vital organs in infants born prematurely.

### SUMMARY OF THE INVENTION

One embodiment of the present invention relates to a method of enhancing organ maturity of premature human neonates by administering to a premature human neonate a growth factor under conditions effective to enhance organ maturation.

Another aspect of the present invention pertains to a method of predicting a premature human neonate's length of stay in a neonatal intensive care unit as well as the anticipated medical costs incurred during said stay. This involves providing a sample from the premature human neonate and determining biomarkers derived from the extracellular matrix in the sample. The biomarker levels or ratios thereof in the sample are compared to a standard to ascertain the premature human neonate's length of stay in a neonatal intensive care unit as well as the anticipated medical costs incurred during said stay.

A further embodiment of the present invention relates to a method of enhancing organ maturity of premature human neonates by administering to a premature human neonate a gene encoding a growth factor under conditions effective to enhance organ maturation.

Another aspect of the present invention is directed to a method of enhancing organ maturity of premature human neonates by administering to a premature human neonate an agent that increases growth factor formation under conditions effective to enhance organ maturation.

An additional aspect of the present invention is directed to a method of increasing collagen type IV formation in a living system by administering to the living system a growth factor, a gene encoding a growth factor, or an agent that increases growth factor formation under conditions effective to increase collagen type IV formation.

In premature neonates, the administration of exogenous growth hormone in the immediate postpartum period is furthermore appropriate since it is known that these patients suffer from a functional deficit of the human Growth Hormone-Insulin-like Growth Factor (“hGH-IGF”) system. This deficiency involves the formation of growth hormone molecules that are detected by conventional immunoassays, yet are inactive when their biological effect is assessed. In premature neonates, immunoreactive growth hormone exceeds bioactive growth hormone by a factor of at least 10 (Radetti et al., “Growth Hormone Bioactivity And Levels Of Growth Hormone, Growth Hormone-Binding Protein, Insulin-Like Growth Factor I, And Insulin-Like Growth Factor-Binding Proteins In Premature And Full-Term Newborns During The First Month Of Life,” Arch. Pediatr. Adolesc. Med., 151:170-175 (1997), which is hereby incorporated by reference in its entirety).

Any method of predicting the length-of-stay in the neonatal intensive care unit (NICU) and the anticipated costs for premature neonates, and any method of advancing the maturation of their epithelial and endothelial surfaces by stimulating synthesis of collagen type IV and thus accelerating basement membrane formation through administration of growth hormone, its various isoforms and analogs, or their associated secondary elements, represents a potentially major advance in the field of pediatrics and heralds remarkable reductions in health care costs. As described above, infants born prematurely (<37 weeks gestational age) account for only 9% of all live births, yet consume 57% of all acute NICU costs in the United States, or $ 5.8 billion annually (St. John et al., “Costs Of Neonatal Care According To Gestational Age At Birth And Survival Status,” Am. J. Obstet. Gynecol., 182:170-175 (2000), which is hereby incorporated by reference in its entirety). This amount accounts for over 40% of total health care costs for all infants. Over the longer term from birth to age 15, follow-up costs due to the medical sequelae of prematurity exceed an additional $ 5.4 billion per year, or 10% of all health care costs for children (Zupancic et al., “Economics Of Prematurity In The Era Of Managed Care,” Clin. Perinatol., 27(2):483-497 (2000), which is hereby incorporated by reference in its entirety).

### BRIEF DESCRIPTION OF THE DRAWINGS

FIGS. 1A-D are graphs showing in four children the typical relationship between the pulsatile nocturnal growth hormone secretion (closed diamonds/coarsely dotted areas) and the matrix biomarker response (closed squares/gradient-shaded areas; FIG. 1A: PICP, procollagen type I C-terminal propeptide; FIG. 1B: C-IV, helical domain of collagen type IV; FIG. 1C: PIIINP, procollagen type III N-terminal propeptide; FIG. 1D: P1 antigen of laminin-1). The physiological rise in morning cortisol secretion (open circles/finely dotted areas) modifies the response in FIGS. 1A and B. Each parameter is, at each time point, expressed as percent deviation from the overall nocturnal mean (%DOM) of that parameter (mean=0% deviation), consistent with standard data presentation in biorhythmic studies.

FIGS. 2A-C are graphs showing that the total amount of growth hormone and the total amount of collagen biomarker (FIG. 2A: PICP, procollagen type I C-terminal propeptide; FIG. 2B: C-IV, helical domain of collagen type IV; FIG. 2C: PIIINP, procollagen type III N-terminal propeptide), both assessed by area-under-curve measurement, display a dose-effect relation.

FIG. 3 is a Kaplan-Meier plot showing the relation between the C-IV biomarker during the first week of life (fw) and the subsequent length-of-stay in the neonatal intensive care unit, whatever the therapeutic interventions after the first 7 days of life. At the cut-off value of less than or equal to 905 ng/ml fwC-IV, the average stay in the NICU is significantly longer than at a cut-off value higher than 905 ng/ml fwC-IV, despite the fact that neonates in either cohort do not differ in mean gestational age (GA). N, number of study subjects.

FIG. 4 is a graph showing the relation between the C-IV biomarker of collagen type IV and the P1 biomarker of laminin, measured in the first week of life (fw), with regard to the subsequent length-of-stay in the NICU. Each data point reflects one premature neonate.

FIG. 5 is a graph showing the mathematical prediction of length-of-stay in the NICU on the basis of fw matrix biomarker measurements, embodied by the ratio [fwC-IV+fwPICP/fwP1]. Each data point reflects one premature neonate.

FIG. 6 is a graph showing the mathematical prediction of length-of-stay in the NICU on the basis of fw matrix biomarker measurements, embodied by the ratio [fwPICP/fwP1]. Each data point reflects one premature neonate.

FIG. 7 is a graph showing the mathematical prediction of length-of-stay in the NICU on the basis of fw matrix biomarker measurements, embodied by fwP1. Each data point reflects one premature neonate.

FIG. 8 shows the absence of any relation between the biomarker parameters presented in FIGS. 5-7 and the gestational age of the neonates.

FIG. 9 is a graph showing the relation between the C-IV biomarker of collagen type IV and the P1 biomarker of laminin, measured in the first week of life (fw), with regard to the subsequent bill for direct physician services rendered in the NICU. Each data point reflects one premature neonate.

FIG. 10 is a graph showing the mathematical prediction of the bills issued for direct physician services rendered in the NICU, the charges of the hospital for supportive services rendered, and the sum of both on the basis of fw biomarker measurements, embodied by the ratio [fwC-IV+fwPICP/fwP1]. The insert shows the absence of any relation between the biomarker parameter and the gestational age of the neonates. Each data point reflects one premature neonate.

FIG. 11 is a graph showing the mathematical prediction of the bills issued for direct physician services rendered in the NICU, the charges of the hospital for supportive services rendered, and the sum of both on the basis of fw biomarker measurements, embodied by the ratio [fwC-IV/fwP1]. The insert shows the absence of any relation between the biomarker parameter and the gestational age of the neonates. Each data point reflects one premature neonate.

FIG. 12 is a graph showing the mathematical prediction of the bills issued for direct physician services rendered in the NICU, the charges of the hospital for supportive services rendered, and the sum of both on the basis of fw biomarker measurements, embodied by fwP1. The insert shows the absence of any relation between the biomarker parameter and the gestational age of the neonates. Each data point reflects one premature neonate.

### DETAILED DESCRIPTION OF THE INVENTION

One embodiment of the present invention relates to a method of enhancing organ maturity of premature human neonates by administering to a premature human neonate a growth factor under conditions effective to enhance organ maturation.

It is well established scientifically, and an integral part of the practice of medicine, that protein factors and hormones (‘mediators’) exert a profound impact on the composition of the human body and on the development and function of its various organs. These protein mediators affect the biology of cells as well as the biology of the extracellular matrix, the composite material in which cells are embedded.

In humans, the most prominent group of these mediators consists of growth hormone itself and of its various isoforms and analogs thought to have evolved from a common precursor, their receptors, and associated secondary elements like the insulin-like growth factors (IGFs). The majority of the growth hormone gene family members are encoded on the long arm of chromosome 17, in the 5′-to-3′ order as follows: growth hormone proper (GH-N)—chorionic somatomammotropin-A (CS-A)—growth hormone variant (GH-V)—chorionic somatomammotropin-B (CS-B). One additional member, prolactin, is encoded on chromosome 6. The IGFs—comprising IGF-1A and -1B, and IGF-2—are encoded on chromosomes 12 and 11, respectively. Numerous binding proteins and receptors for these mediators are known. The physiology and pathology of the hGH-IGF system and their practical and economic ramifications are the topic of several recent reviews (Bercu et al., “Growth Hormone: Relevance To Pediatrics,” In: Human Growth Hormone. Research And Clinical Practice, Smith et al., Eds., Humana Press, Totowa, pp. 191-220 (2000); Carroll et al., “Growth Hormone Deficiency In Adults: The Rationale For Growth Hormone Replacement,” In: Human Growth Hormone. Research And Clinical Practice. Smith et al., Eds., Humana Press, Totowa, pp. 221-232 (2000); Rosenfeld et al., “Disorders Of Growth Hormone/Insulin-Like Growth Factor Secretion And Action,” In: Pediatric Endocrinology, Sperling, Ed., Saunders, Philadelphia, pp. 211-288 (2002); and Kopchick, “Growth Hormone,” In: Endocrinology, DeGroot et al., Eds., Saunders, Philadelphia, pp. 389-404 (2001), which are hereby incorporated by reference in their entirety).

The extracellular matrix is one the main targets of growth hormone, the members of the growth hormone gene family, and of associated secondary elements like the IGFs. The deficiency of adequate production and secretion of growth hormone invariably leads to severe short stature in childhood, a period in the human life cycle when growth hormone assumes the role of main mediator for height development. Earlier in life, growth hormone is a key factor for metabolic control, e.g. of blood sugar levels, and later in life, growth hormone contributes to maintaining bone density and to avert osteoporosis (Bercu et al., “Growth Hormone: Relevance To Pediatrics,” In: Human Growth Hormone. Research And Clinical Practice, Smith et al., Eds., Humana Press, Totowa, pp. 191-220 (2000); Rosenfeld et al., “Disorders Of Growth Hormone/Insulin-Like Growth Factor Secretion And Action,” In: Pediatric Endocrinology, Sperling, Ed., Saunders, Philadelphia, pp. 211-288 (2002); and Baum et al., “Growth Hormone And Osteoporosis. In: Human Growth Hormone. Research And Clinical Practice,” Smith et al., Eds., Humana Press, Totowa, pp. 233-246 (2000), which are hereby incorporated by reference in their entirety).

Consistent with this activity profile, growth hormone drives the formation of those collagens that are prominent in the growth plate of bones, and in bone itself. Among the nineteen different types of collagens, the major protein components of the extracellular matrix, three types occur particularly prominently at these anatomical sites and were found to be reactive to growth hormone and the IGFs. These are collagen type I, collagen type III, and collagen type X (for review on collagens type I and type III, see Vihervuori et al., “Collagen Formation And Degradation Increase During Growth Hormone Therapy In Children,” Bone, 20:133-138 (1997), which is hereby incorporated by reference in its entirety, and literature cited therein; for collagen type X, see Kikkawa et al., “Altered Postnatal Expression Of Insulin-Like Growth Factor I (IGF-I) And Type X Collagen Preceding The Perthes' Disease-Like Lesion Of A Rat Model,” J. Bone Miner. Res., 15:111-119 (2000), which is hereby incorporated by reference in its entirety). In fact, the matrix formation-enhancing activity of growth hormone is so pronounced that the abuse of growth hormone for enhancement of athletic performance can be detected by the measurement of biomarkers that originate from the extracellular matrix, in particular collagen type III (Langobardi et al., “Growth Hormone (GH) Effects On Bone And Collagen Turnover In Healthy Adults And Its Potential As A Marker Of GH Abuse In Sports: A Double-Blind, Placebo-Controlled Study,” J. Clin. Endocrinol. Metab., 85:1505-1512 (2000), which is hereby incorporated by reference in its entirety). As a medication, recombinantly produced growth hormone is routinely used to enhance the formation of collagens type I, III, and X, and thus improve height and bone structure in several disease states defined by growth disorders, such as endogenous growth hormone deficiency or genetically and iatrogenically caused forms of short stature (Rosenfeld et al., “Disorders Of Growth Hormone/Insulin-Like Growth Factor Secretion And Action,” In: Pediatric Endocrinology, Sperling, Ed., Saunders, Philadelphia, pp. 211-288 (2002), which is hereby incorporated by reference in its entirety). Growth hormone also shows promising results in the treatment of several catabolic states, e.g. due to aging or to infection with human immunodeficiency virus (Baum et al., “Growth Hormone And Osteoporosis. In: Human Growth Hormone. Research And Clinical Practice,” Smith et al., Eds., Humana Press, Totowa, pp. 233-246 (2000) and Mulligan et al., “Growth Hormone And AIDS. In: Human Growth Hormone. Research And Clinical Practice,” Smith et al., eds. Humana Press, Totowa, pp. 285-296 (2000), which are hereby incorporated by reference in their entirety). As exemplified by endogenous growth hormone deficiency, lack of adequate growth hormone secretion is associated with low levels of biomarkers derived from collagens type I and type III. Conversely, administration of exogenous growth hormone causes the biomarkers derived from collagens type I and type III to increase markedly (for review, see Vihervuori et al., “Collagen Formation And Degradation Increase During Growth Hormone Therapy In Children,” Bone, 20:133-138 (1997), which is hereby incorporated by reference in its entirety, and literature cited therein).

Thus, in accordance with the present invention, suitable growth factors include, but are not limited to, endocrine growth factors, embodied by those encoded on human chromosome 17, on chromosome 12, on chromosome 11, and on chromosome 6. In particular, suitable growth factors encoded on human chromosome 17 include hGH-N, hGH-V, chorionic somatomammotropin-A, and chorionic somatomammotropin-B. Suitable growth factors encoded on human chromosome 12 include IGF-1. Suitable growth factors encoded on human chromosome 11 include IGF-2. Suitable growth factors encoded on human chromosome 6 include prolactin.

Also, in accordance with the present invention suitable growth factors include the group of matrix growth factors, embodied by the laminins that are encoded on various human chromosomes.

The growth factors are administered to premature human neonates under conditions effective to enhance organ maturation. In particular, the growth factors are administered under conditions effective to increase basement membranes in organs of premature human neonates. Further, the growth factors are administered under conditions effective to increase collagen type IV formation in organs of premature human neonates.

More specifically, we have found that in humans not only collagens type I and type III, but also collagen type IV is under the control of the hGH-IGF system. Whereas collagens type I and type III form fibrils and fibers, and thus are prominent in tissues exposed to biomechanical stress, collagen type IV does not form fibrils or fibers. Instead, this collagen forms a sheet-like polymer that is the main component of basement membrane, the structure which anchors the cells that form the epithelial surfaces of lung, gut, and kidney, and that form the endothelial surfaces inside of the cardiovascular system (Hanauske-Abel, “Fibrosis Of The Liver: Representative Molecular Elements, And Their Emerging Role As Antifibrotic Targets,” In: Hepatology. A Textbook of Liver Disease, Zakim et al., Eds., Saunders, Philadelphia (2002) (in press), which is hereby incorporated by reference in its entirety). This finding indicates that if a human condition exists that is associated with low biomarker levels for collagen type IV, then this condition should be relieved by the exogenous administration of growth hormone, its various isoforms and analogs, or their associated secondary elements, especially if this condition coincides with functional deficiencies in the hGH-IGF system. We have unexpectedly identified such a condition in a unique class of patients.

Growth hormone is secreted into the blood stream in a pulsatile manner, particularly during sleep (Bercu et al., “Growth Hormone: Relevance To Pediatrics,” In: Human Growth Hormone. Research And Clinical Practice, Smith et al., Eds., Humana Press, Totowa, pp. 191-220 (2000) and Rosenfeld et al., “Disorders Of Growth Hormone/Insulin-Like Growth Factor Secretion And Action,” In: Pediatric Endocrinology, Sperling, Ed., Saunders, Philadelphia, pp. 211-288 (2002), which are hereby incorporated by reference in their entirety). This discontinuous secretion allows monitoring the in vivo response of the extracellular matrix by measuring serum biomarkers that are specific for either the collagenous or the non-collagenous proteins of the matrix. Each nocturnal growth hormone peak should coincide with, or be followed by, an appropriately timed peak of a matrix biomarker, provided the formation of this biomarker is responsive to growth hormone. If it is not, then the vast and rapid nocturnal fluctuations of the growth hormone level are dissociated from the level of the matrix biomarker. This general relationship between growth hormone stimulus and matrix response should even apply in the morning hours, when the secretion of cortisol becomes activated, a powerful antagonist of growth hormone and generally known to inhibit matrix formation.

Thus, organ maturation can be achieved in organs, such as the lungs, kidneys, gastrointestinal tract, heart and skeletal muscle, and blood vessels. In one particular embodiment, organ maturation is achieved in the lungs. In accordance with the present invention, the growth factor is administered in a dose sufficient to enhance organ maturation.

The growth factor can be administered by inhalation, orally, subcutaneously, intravenously, intramuscularly, intraperitoneally, or by application to mucous membranes, such as, that of the nose, throat, and bronchial tubes. In one particular embodiment, when organ maturation is to be achieved in the lungs, administration of the growth factor is carried out by inhalation. The growth factor can be administered alone or with pharmaceutically or physiologically acceptable carriers, excipients, or stabilizers, and can be in solid or liquid form such as, powders, solutions, suspensions, or emulsions.

The solid unit dosage forms can be of the conventional type. The solid form can be a capsule, such as an ordinary gelatin type containing the growth factor and a carrier, for example, lubricants and inert fillers, such as lactose, sucrose, or cornstarch. In another embodiment, these growth factors can be tableted with conventional tablet bases, such as lactose, sucrose, or cornstarch, in combination with binders, like acacia, cornstarch, or gelatin, disintegrating agents, such as cornstarch, potato starch, or alginic acid, and lubricants, like stearic acid or magnesium stearate.

The growth factor may also be administered in injectable dosages by solution or suspension of this material in a physiologically acceptable diluent with a pharmaceutical carrier. Such carriers include sterile liquids, such as water and oils, with or without the addition of a surfactants, adjuvants, excipients, or stabilizers. Illustrative oils are those of petroleum, animal, vegetable, or synthetic origin, for example, peanut oil, soybean oil, or mineral oil. In general, water, saline, aqueous dextrose and related sugar solutions, and glycols, such as propylene glycol or polyethylene glycol, are preferred liquid carriers, particularly for injectable solutions.

For use as aerosols, the growth factor in solution or suspension may be packaged in a pressurized aerosol container together with suitable propellants, for example, hydrocarbon propellants like propane, butane, or isobutane, and with conventional adjuvants. The growth factor can also be administered in a non-pressurized form, such as in a nebulizer or atomizer.

The present invention also relates to a method of predicting a premature human neonate's length of stay in a neonatal intensive care unit as well as the anticipated medical costs incurred during said stay. This method involves providing a sample from the premature human neonate and determining biomarkers derived from the extracellular matrix in the sample. The biomarker levels or ratios thereof in the sample are compared to a standard to ascertain the premature human neonate's length of stay in a neonatal intensive care unit as well as the anticipated medical costs incurred during said stay.

Suitable samples include biological samples, such as plasma, serum, urine, bronchial lavage liquid, and swabs of bodily fluids.

In accordance with one embodiment of the present invention, the biomarkers derived from the extracellular matrix originate from collagenous proteins. In a particular embodiment, the biomarkers derived from the extracellular matrix originate from collagen type IV in organs of premature human neonates.

In another embodiment of the present invention, the biomarkers derived from the extracellular matrix originate from non-collagenous proteins or glycosaminoglycans. In a particular embodiment, the biomarkers derived from the extracellular matrix originate from laminin in organs of premature human neonates.

The biomarkers may be determined using methods known to those of ordinary skill in the art. In particular, collagenous and non-collagenous biomarkers of the extracellular matrix can be determined in any biological fluid by commercially available, highly specific and reproducible immunoassays and/or chromatographic procedures (see, e.g., Aghai et al., “Basement Membrane Biomarkers in Very Low Birth Weight Premature Infants,” Biol. Neonate, 81:16-22 (2002), which is hereby incorporated by reference in its entirety). These products can be purchased by and reliably applied in any clinical laboratory by those of ordinary skill in clinical chemistry. Once the biomarkers in the sample are determined, the levels of biomarkers or ratios thereof are used in a mathematical formula to estimate the premature human neonate's length of stay in the NICU as well as the anticipated costs incurred during the stay. An appropriately accurate mathematical formula (‘standard’) for each biomarker or biomarker ratio is developed by routine statistical analysis of the relation between the biomarker parameter in premature neonates and their length-of-stay in the NICU, or their NICU costs incurred, respectively, as described below, e.g., in Example 2

A further embodiment of the present invention relates to a method of enhancing organ maturity of premature human neonates by administering to a premature human neonate a gene encoding a growth factor under conditions effective to enhance organ maturation.

In accordance with this aspect of the present invention, the gene encoding the growth factor is selected from the group consisting of genes encoding endocrine growth factors, embodied by those encoded on human chromosome 17, on chromosome 12, on chromosome 11, and on chromosome 6. In one particular embodiment, the growth factor is encoded on human chromosome 17 and is selected from the group consisting of hGN-N, hGH-V, chorionic somatomammotropin-A, and chorionic somatomammotropin-B. In another particular embodiment, the growth factor is encoded on human chromosome 12 and is IGF-1. In yet another embodiment, the growth factor is encoded on human chromosome 11 and is IGF-2. In a further embodiment, the growth factor is encoded on human chromosome 6 and is prolactin. In still a further embodiment, the gene encoding the growth factor encodes a matrix growth factor, embodied by the laminins that are located on various human chromosomes.

A gene or cDNA encoding the desired growth factor, or a fragment thereof, may be obtained, for example, by screening a genomic or cDNA library, or by PCR amplification.

A gene encoding a growth factor may be administered in a nucleic acid construct, which involves incorporating nucleic acid molecules into host cells using conventional recombinant DNA technology. Generally, this involves inserting the nucleic acid molecule into an expression system to which the nucleic acid molecule is heterologous (i.e., not normally present). The heterologous nucleic acid molecule is inserted into the expression system which includes the necessary elements for the transcription and translation of the inserted coding sequences. The practice of the present invention will employ, unless otherwise indicated, conventional methods of virology, microbiology, molecular biology, and recombinant DNA techniques within the skill of the art. Such techniques are explained fully in the literature. See, e.g., Sambrook, et al., Molecular Cloning: A Laboratory Manual (1989); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Fundamental Virology, 2nd Edition, vol. I & II (B. N. Fields and D. M. Knipe, eds.), which are hereby incorporated by reference in their entirety.

The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The nucleic acid molecules may be inserted into any of the many available expression vectors and cell systems using reagents that are well known in the art.

Examples of viruses which have been employed as vectors for the transduction and expression of exogenous genes in mammalian cells include the SV40 virus (Innis et al., “Chromatin Structure of Simian Virus 40-pBR322 Recombinant Plasmids in COS-1 Cells,” Mol. Cell Biol. 3(12):2203-2210 (1983); Okayama et al., “Bacteriophage Lambda Vector for Transducing a cDNA Clone Library into Mammalian Cells,” Mol. Cell Biol. 5(5):1136-1142 (1985), which are hereby incorporated by reference in their entirety) and bovine papilloma virus (Meneguzzi et al., “Plasmidial Maintenance in Rodent Fibroblasts of a BPV1-pBR322 Shuttle Vector Without Immediately Apparent Oncogenic Transformation of the Recipient Cells,” EMBO J. 3(2):365-371 (1984); DiMaio et al., “Bovine Papillomavirus Vector that Propagates as a Plasmid in Both Mouse and Bacterial Cells,” Proc. Nat'l. Acad. Sci. USA 79(13):4030-4034 (1982); Lusky et al., “Characterization of the Bovine Papilloma Virus Plasmid Maintenance Sequences,” Cell 36(2):391-401 (1984); Giri et al., “Comparative Studies of the Expression of Linked Escherichia coli gpt Gene and BPV-1 DNAs in Transfected Cells,” Virology 127(2):385-396 (1983), which are hereby incorporated by reference in their entirety), the retrovirus Moloney murine sarcoma virus (Perkins et al., “Design of a Retrovirus-Derived Vector for Expression and Transduction of Exogenous Genes in Mammalian Cells,” Mol. Cell Biol. 3(6): 1123-1132 (1983); Lee et al., “DNA Clone of Avian Fujinami Sarcoma Virus with Temperature-Sensitive Transforming Function in Mammalian Cells,” J. Virol. 44(1):401-412 (1982); Curran et al., “FBJ Murine Osteosarcoma Virus: Identification and Molecular Cloning of Biologically Active Proviral DNA,” J. Virol. 44(2):674-682 (1982); Gazit et al., “Mammalian Cell Transformation by a Murine Retrovirus Vector Containing the Avian Erythroblastosis Virus erbB Gene,” J. Virol. 60(1):19-28 (1986), which are hereby incorporated by reference in their entirety), and HIV-based viruses.

A number of adenovirus (Ad) based gene delivery systems have also been developed. Human adenoviruses are double-stranded DNA viruses which enter cells by receptor-mediated endocytosis. These viruses are particularly well suited for gene therapy, because they are easy to grow and manipulate and they exhibit a broad host range in vivo. Adenovirus is easily produced at high titers and is stable so that it can be purified and stored. Even in the replication-competent form, adenoviruses generally cause only low level morbidity and are not associated with human malignancies. Furthermore, Ad infects both dividing and non-dividing cells; a number of tissues which are targets for gene therapy comprise largely non-dividing cells (U.S. Pat. No. 6,171,855 to Askari, which is hereby incorporated by reference in its entirety). For descriptions of various adenovirus-based gene delivery systems, see, e.g., Haj-Ahmad et al., “Development of a Helper-Independent Human Adenovirus Vector and Its Use in the Transfer of the Herpes Simplex Virus Thymidine Kinase Gene,” J. Virol. 57(1):267-274 (1986); Bett et al., “Packaging Capacity and Stability of Human Adenovirus Type 5 Vectors,” J. Virol. 67(10):5911-5921 (1993); Mittereder et al., “Evaluation of the Efficacy and Safety of in vitro, Adenovirus-Mediated Transfer of the Human Cystic Fibrosis Transmembrane Conductance Regulator cDNA,” Hum. Gene Ther. 5(6):717-729 (1994); Seth et al., “Mechanism of Enhancement of DNA Expression Consequent to Cointernalization of a Replication-Deficient Adenovirus and Unmodified Plasmid DNA,” J. Virol. 68(2):933-940 (1994); Barr et al., “Efficient Catheter-Mediated Gene Transfer into the Heart Using Replication-Defective Adenovirus,” Gene Ther. 1(1):51-58 (1994); Berkner et al., “Development of Adenovirus Vectors for the Expression of Heterologous Genes,” Biotechniques 6(7):616-629 (1988); Rich et al., “Development and Analysis of Recombinant Adenoviruses for Gene Therapy of Cystic Fibrosis,” Hum. Gene Ther. 4(4):461-476 (1993), which are hereby incorporated by reference in their entirety.

Retroviral vectors, capable of integration into the cellular chromosome, have also been used for the identification of developmentally important genes via insertional mutagenesis (see, e.g., U.S. Pat. No. 6,207,455 to Chang, which is hereby incorporated by reference in its entirety). Retroviral vectors are also used in therapeutic applications (e.g., gene therapy), in which a gene (or genes) is added to a cell to replace a missing or defective gene or to inactivate a pathogen such as a virus. The members of the family Retroviridae are characterized by the presence of reverse transcriptase in their virions (U.S. Pat. No. 6,207,344 to Chang, which is hereby incorporated by reference in its entirety). The family is divided into three subfamilies: (1) Oncovirinae, including all the oncogenic retroviruses, and several closely related non-oncogenic viruses; (2) Lentivirinae, the “slow retroviruses,” discussed in greater detail below, and (3) Spumavirinae, the “foamy” retroviruses that induce persistent infections, generally without causing any clinical disease (U.S. Pat. No. 6,218,181 to Verma et al., which is hereby incorporated by reference in its entirety). Some of the retroviruses are oncogenic (i.e., tumorigenic), while others are not. The oncoviruses induce sarcomas, leukemias, lymphomas, and mammary carcinomas in susceptible species (U.S. Pat. No. 6,033,905 to Wilson et al., which is hereby incorporated by reference in its entirety). Retroviruses infect a wide variety of species, and may be transmitted both horizontally and vertically. They are integrated into the host DNA, and are capable of transmitting sequences of host DNA from cell to cell. This has led to the development of retroviruses as vectors for various purposes, including gene therapy. For example, the majority of the approved gene transfer trials in the United States rely on replication-defective retroviral vectors harboring a therapeutic polynucleotide sequence as part of the retroviral genome (Miller et al., “Gene Transfer by Retrovirus Vectors Occurs Only in Cells that are Actively Replicating At The Time of Infection,” Mol. Cell Biol. 10(8):4239-4442 (1990); Cornetta et al., “No Retroviremia or Pathology in Long-term Follow-up of Monkeys Exposed to Amphotropic Retrovirus,” Hum. Gene Ther. 2(3):215-219 (1991), which are hereby incorporated by reference in their entirety). As is known in the art, the major advantages of retroviral vectors for gene therapy are the high efficiency of gene transfer into certain types of replicating cells, the precise integration of the transferred genes into cellular DNA, and the lack of further spread of the sequences after gene transfer (U.S. Pat. No. 6,033,905 to Wilson et al., which is hereby incorporated by reference in its entirety).

As used herein, the term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells). Lentivirus virions have bar-shaped nucleoids and contain genomes that are larger than other retroviruses. Lentiviruses use tRNA^{lys} as primer for negative-strand synthesis, rather than the tRNA^{pro} commonly used by other infectious mammalian retroviruses. The lentiviral genomes exhibit homology with each other, but not with other retroviruses (Davis et al., Microbiology, 4th ed., J. B. Lippincott Co., Philadelphia, Pa., pp. 1123-1151 (1990), which is hereby incorporated by reference in its entirety). An important factor in the disease caused by these! viruses is the high mutability of the viral genome, which results in the production of mutants capable of evading the host immune response. The advantage of lentiviruses is the ability for sustained transgene expression.

Adeno-associated viruses (AAV) may also be employed as a vector in the present invention. AAV is a small, single-stranded (ss) DNA virus with a simple genomic organization (4.7 kb) that makes it an ideal substrate for genetic engineering. Two open reading frames encode a series of rep and cap polypeptides. Rep polypeptides (rep78, rep68, rep62, and rep40) are involved in replication, rescue, and integration of the AAV genome. The cap proteins (VP1, VP2, and VP3) form the vinon capsid. Flanking the rep and cap open reading frames at the 5′ and 3′ ends are 145 bp inverted terminal repeats (ITRs), the first 125 bp of which are capable of forming Y- or T-shaped duplex structures. Of importance for the development of AAV vectors, the entire rep and cap domains can be excised and replaced with a therapeutic or reporter transgene (B. J. Carter, in “Handbook of Parvoviruses”, ed., P. Tijsser, CRC Press, pp. 155-168 (1990), which is hereby incorporated by reference in its entirety). It has been shown that the ITRs represent the minimal sequence required for replication, rescue, packaging, and integration of the AAV genome (U.S. Pat. No. 5,871,9982 to Wilson et al., which is hereby incorporated by reference in its entirety).

As noted above, viral vectors have been successfully employed in order to increase the efficiency of introducing a recombinant vector into suitably sensitive host cells. Therefore, viral vectors are suited for use in the present invention, including any adenoviral (Ad), retroviral, lentiviral, or adeno-associated viral (AAV) vectors described above or known in the art. Current research in the field of viral vectors is producing improved viral vectors with high-titer and high-efficiency of transduction in mammalian cells (see, e.g., U.S. Pat. No. 6,218,187 to Finer et al., which is hereby incorporated by reference in its entirety). Such vectors are suitable in the present invention, as is any viral vector that comprises a combination of desirable elements derived from one or more of the viral vectors described herein. It is not intended that the expression vector be limited to a particular viral vector.

Certain “control elements” or “regulatory sequences” are also incorporated into the vector-construct. The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription, and translation of a coding sequence in a recipient cell. Not all of these control elements need always be present so long as the selected coding sequence is capable of being replicated, transcribed, and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcriptional control signals in eukaryotes comprise “promoter” and “enhancer” elements. Promoter and enhancer elements have been isolated from a variety of eukaryotic sources, including genes in yeast, insect, and mammalian cells, and viruses. Analogous control elements, i.e., promoters, are also found in prokaryotes. Such elements may vary in their strength and specificity. For example, promoters may be “constitutive” or “inducible.”

A constitutive promoter is a promoter that directs expression of a gene throughout the development and life of an organism. Examples of some constitutive promoters that are widely used for inducing expression of transgenes include the nopoline synthase (NOS) gene promoter from Agrobacterium tumefaciens (U.S. Pat. No. 5,034,322 to Rogers et al., which is hereby incorporated by reference in its entirety), the cytomegalovirus (CMV) early promoter, those derived from any of the several actin genes, which are known to be expressed in most cells types (U.S. Pat. No. 6,002,068 to Privalle et al., which is hereby incorporated by reference in its entirety), and the ubiquitin promoter, which is a gene product known to accumulate in many cell types.

An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent, such as a metabolite, or a physiological stress directly imposed upon the organism such as cold, heat, toxins, or through the action of a pathogen or disease agent. A recombinant cell containing an inducible promoter may be exposed to an inducer by externally applying the inducer to the cell or organism by exposure to the appropriate environmental condition or the operative pathogen. Examples of inducible promoters include the tetracycline response element and promoters derived from the 13-interferon gene, heat shock gene, metallothionein gene or any obtainable from steroid hormone-responsive genes. Tissue specific expression has been well characterized in the field of gene expression and tissue specific and inducible promoters are well known in the art. These genes are used to regulate the expression of the foreign gene after it has been introduced into the target cell.

Constitutive or inducible promoters may be used in the viral vectors of this invention.

The vector of choice, a suitable marker gene, promoter/enhancer region(s), and an appropriate 3′ regulatory region can be operably ligated together to produce the expression system of the present invention, or suitable fragments thereof, using well known molecular cloning techniques as described in Sambrook et al., Molecular Cloning: A Laboratory Manual, Second Edition, Cold Spring Harbor Press, NY (1989), and Ausubel et al. (1989) Current Protocols in Molecular Biology, John Wiley & Sons, New York, N.Y., which are hereby incorporated by reference in their entirety. The term “operably linked” as used herein refer to the linkage of nucleic acid sequences in such a manner that a nucleic acid molecule capable of directing the transcription of a given gene and/or the synthesis of a desired protein molecule is produced.

Once the nucleic acid construct of the present invention has been prepared and inserted into the desired vector, it is ready to be incorporated into a host cell. Basically, this method is carried out by transforming a host cell with a nucleic construct of the present invention under conditions effective to yield transcription of the DNA molecule in the host cell, using standard cloning procedures known in the art, such as that described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, mammalian cells, insect, plant, and the like. Where the vector is a viral vector, the host cell is chosen to optimize packaging, where required, and titer. For example, where the nucleic acid of the present invention is inserted into an adenovirus vector, the cell line HEK293 is an appropriate host line, with the expectation of high vector progeny titers. The vector DNA may be introduced into the packaging cell by any of a variety of transfection techniques, e.g., calcium phosphate coprecipitation, electroporation, etc. (See, e.g., Sambrook, et al., Molecular Cloning: A Laboratory Manual (1989); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Fundamental Virology, 2nd Edition, vol. I & II (B. N. Fields and D. M. Knipe, eds.), which are hereby incorporated by reference in their entirety.) Other conventional methods employed in this invention include homologous recombination of the viral genomes, plaquing of viruses in agar overlay, methods of measuring signal generation, and the like known in the art or described in literature.

Following transfection of an appropriate host with the viral vector of the present invention, the virus is propagated in the host and collected. Generally, this involves collecting the cell supernatants at periodic intervals, and purifying the viral plaques from the crude lysate, using techniques well-known in the art, for example, cesium chloride density gradient. The titer (pfu/ml) of the virus is determined, and can be adjusted up (by filtration, for example), or down (by dilution with an appropriate buffer/medium), as needed. In the present invention, typical Ad titers are in the range of 10¹⁰-10¹² pfu/ml.

To effect the gene therapy aspect of the present invention, the isolated, purified viral vector containing the growth factor-encoding nucleic acid or human cells infected with same, is administered to a premature human neonate under conditions effective to express the growth factor and to enhance organ maturation in the neonate.

The recombinant viruses of the present invention may be administered to a neonate, preferably suspended in a biologically compatible solution or pharmaceutically acceptable delivery vehicle or in any cell type derived from said neonate. A suitable vehicle includes sterile saline. Other aqueous and non-aqueous isotonic sterile solutions and aqueous and non-aqueous sterile suspensions known to be pharmaceutically acceptable carriers and well known to those of skill in the art may be employed for this purpose.

The recombinant viruses of this invention may be administered in sufficient amounts to transfect the desired cells in vitro or in vivo and provide sufficient levels of integration and expression of the selected transgene to provide a therapeutic benefit without undue adverse effects or with medically acceptable physiological effects which can be determined by those skilled in the medical arts. While the preferable method of administration is by inhalation when enhancing lung maturation, other conventional and pharmaceutically acceptable methods of administration including intravenous, intramuscular, subcutaneous, intraperitoneal, application to mucous membranes, and oral administration are encompassed by the present invention.

Dosages of the recombinant virus will depend primarily on factors, such as the selected gene, the age, weight, and health of the patient, and may thus vary among patients. The dosage will be adjusted to balance the therapeutic benefit against any side effects. The levels of expression of the selected gene can be monitored to determine the selection, adjustment, or frequency of dosage administration.

Another aspect of the present invention is directed to a method of enhancing organ maturity of premature human neonates by administering to a premature human neonate an agent that increases growth factor formation under conditions effective to enhance organ maturation.

Suitable agents that increase growth factor formation include, but are not limited to, growth hormone releasing hormone and its peptide analogs, and peptidomimetic growth hormone secretagogues, as exemplified by MK-0677 (Patchett et al., “The Design of Peptidomimetic Growth Hormone Secretagogues,” In: Human Growth Hormone. Research and Clinical Practice, Smith et al., Eds., Humana Press, Totowa, pp. 45-67 (2000), which is hereby incorporated by reference in its entirety).

An additional aspect of the present invention is directed to a method of increasing collagen type IV formation in a living system by administering to the living system a growth factor, a gene encoding a growth factor, or an agent that increases growth factor formation under conditions effective to increase collagen type IV formation.

As used herein, a living system is meant herein to include any member of the class Mammalia including, without limitation, humans and non-human primates, such as chimpanzees and other apes and monkey species; farm animals including cattle, sheep, pigs, goats and horses; domestic animals including cats and dogs; laboratory animals including rodents such as mice rats, and guinea pigs, and the like. The term does not denote a particular age or sex. Thus, adults and neonates, as well as fetuses, are intended to be covered.

The following examples further illustrate the present invention.

### EXAMPLES

### Example 1

### Relation Between Nocturnal Growth Hormone and Matrix Biomarker Levels

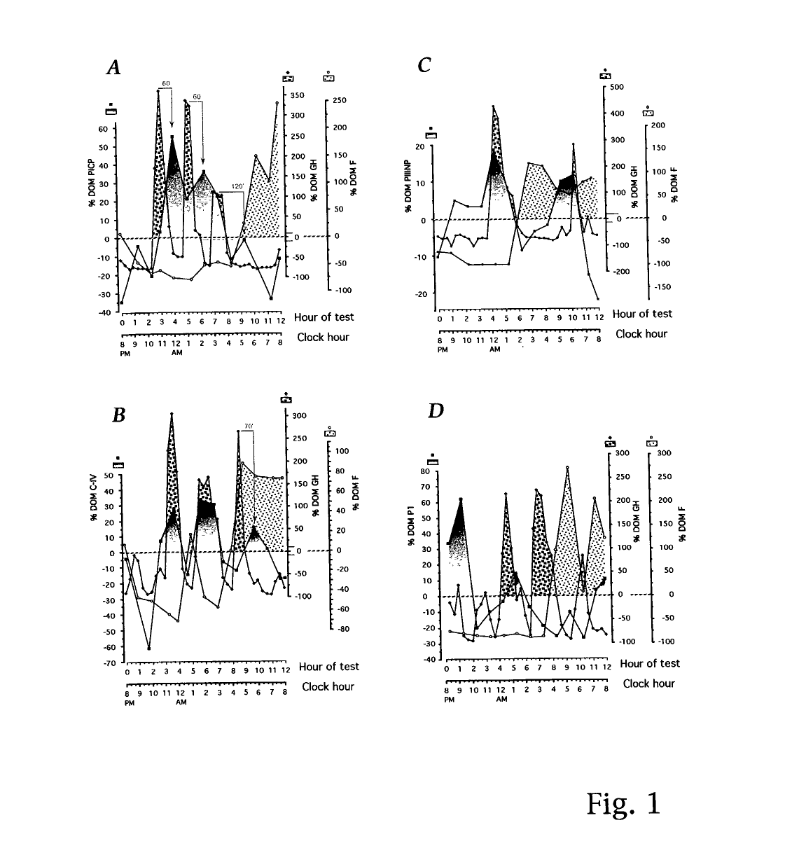

With parental informed consent, minor assent, and institutional approval, the relation between nocturnal growth hormone and matrix biomarker levels in healthy children was monitored, drawing blood samples every 20 minutes during their sleep. All parameters were measured by specific, standardized, and generally accepted methods in the serum obtained from the blood draws. Representative findings are shown in the triple y-axis plots of FIGS. 1A-D. In each plot, the horizontal axes indicate time (hour of test and clock hour), the left vertical axis gives the level of the matrix biomarker, and the two vertical axes on the right show the levels of growth hormone and of cortisol, respectively. Matrix biomarker fluctuations are encoded by closed squares/gradient-shaded areas (PICP, biomarker for collagen type I; C-IV, biomarker for collagen type IV; PIIINP, biomarker for collagen type III; PI, biomarker for laminin), fluctuations of growth hormone (GH) by closed diamonds/coarsely dotted areas, and fluctuations of cortisol (F) by open circles/finely dotted areas. Following established procedure in the field of biorhythm studies, the value for a parameter at a given time is expressed as its percent deviation from the parameter's overall mean during the observation period (percent deviation from the mean, or %DOM). FIG. 1A displays the interrelation between collagen type I, per PICP, and GH and F. FIG. 1B displays the interrelation between collagen type IV, per C-IV, and GH and F. FIG. 1C displays the interrelation between collagen type III, per PIIINP, and GH and F. FIG. 1D displays the interrelation between laminin, per PI, and GH and F. As expected, all the peaks in the biomarker levels for collagens type I and type III (coarsely dotted areas/gradient-shaded areas in FIGS. 1A and 1C) consistently relate to growth hormone peaks. Surprisingly, growth hormone also triggers peaks in the biomarker levels for collagen type IV (coarsely dotted areas/gradient-shaded areas in FIG. 1B), indicating that the biosynthesis of basement membrane collagen increases if the growth hormone concentration increases, and reaches its maxima as the growth hormone levels reach theirs. By contrast, the synthesis of laminin does not correlate with growth hormone values (coarsely dotted areas/gradient-shaded areas in FIG. 1D), although laminin and collagen type IV are similarly required for the formation of basement membrane.

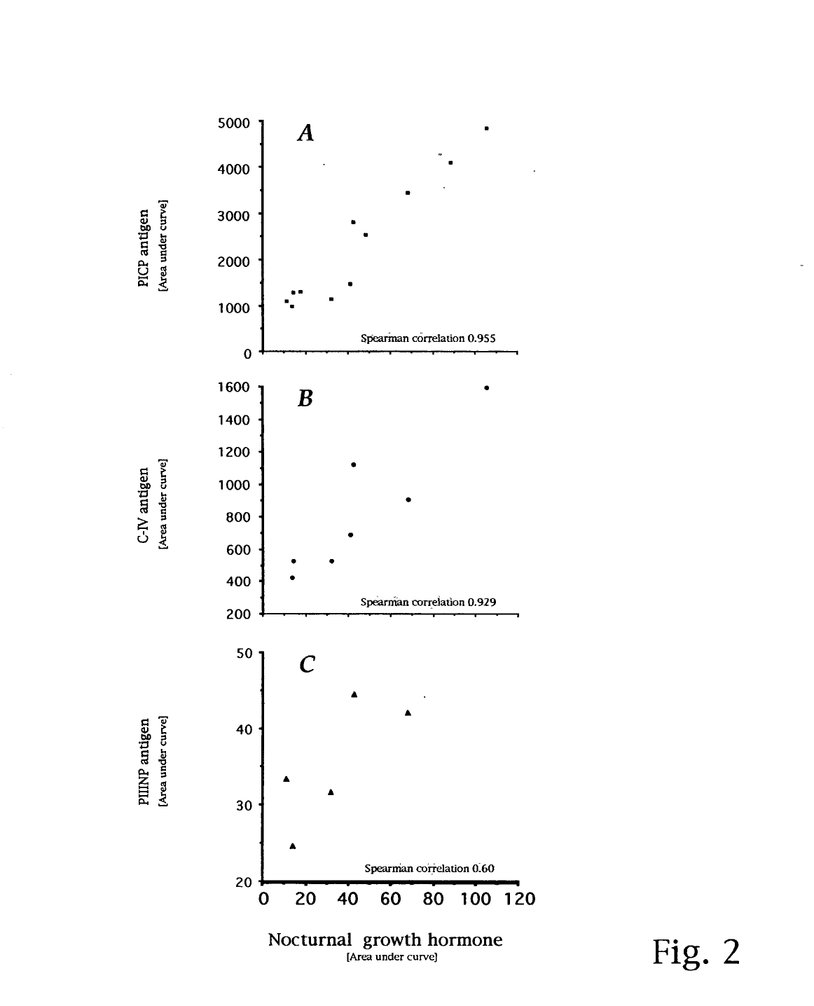

FIGS. 2A-C show per study subject that the total amount of growth hormone and the total amount of collagen biomarker, both assessed by area-under-curve measurement, display a dose-effect relation: the lower growth hormone, the lower the biomarker (FIG. 2A: PICP for collagen type I; FIG. 2B: C-IV for collagen type IV; FIG. 2C: PIIINP for collagen type III). In summary, endogenous human growth hormone in vivo stimulates the formation of collagen type IV, in addition to stimulating collagen type I and type III. Thus, administration of exogenous human growth hormone qualifies for the alleviation of any condition characterized by a deficiency in collagen type IV synthesis, just like growth hormone qualifies for the alleviation of any condition characterized by a deficiency in collagen type I and type III synthesis.

### Example 2

### Relation Between Matrix Biomarkers in the First Week of Life and Various Clinical Parameters of Their Subsequent Course

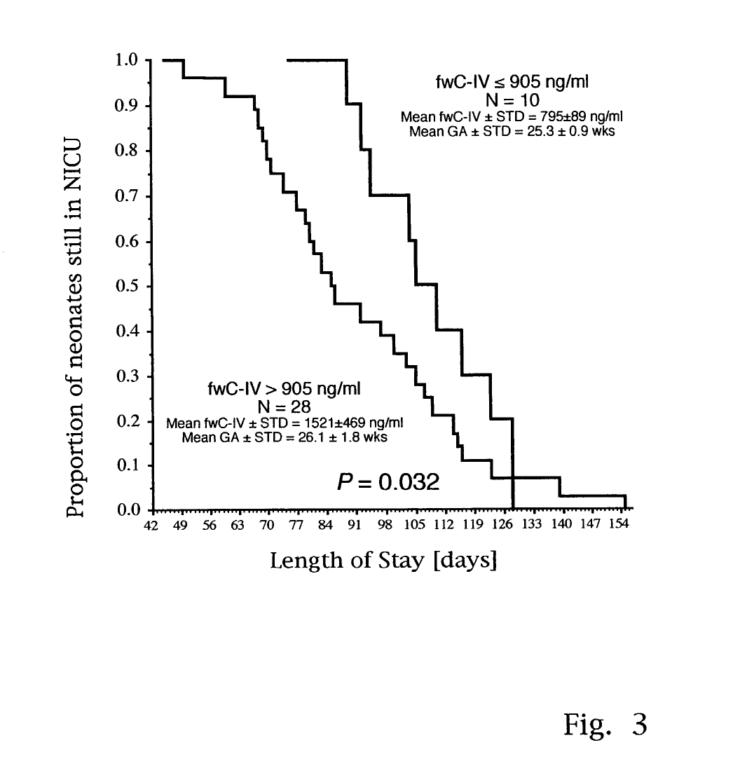

With parental informed consent and institutional approval, in a group of premature neonates the relation between matrix biomarkers in the first week of life (fw) and various clinical parameters of their subsequent course, whatever the therapeutic interventions, was monitored. As shown by the Kaplan-Meier analysis in FIG. 3, those premature neonates that had fwC-IV levels of less than or equal to 905 ng/ml required a significantly longer admission to the neonatal intensive care unit than those whose fwC-IV levels were above 905 ng/ml: 110 days versus 85 days, respectively. The mean gestational age between both groups did not differ. Similar cut-off values could not be defined for any other matrix biomarker. It is entirely unanticipated that a biomarker should exist that, when measured in premature neonates during the first week of life, significantly correlates with the length-of-stay in neonatal intensive care that evolves subsequently, only long after the first week of life. FIG. 3 also teaches that any drug that increases fwC-IV, i.e. shifts the curve on the right towards the one on the left, can be reasonably expected to shorten the length-of-stay in neonatal intensive care.

Since the length of stay in the neonatal intensive care unit (NICU) is known to determine the neonatal intensive care unit costs incurred by survivors (St. John et al., “Costs Of Neonatal Care According To Gestational Age At Birth And Survival Status,” Am. J. Obstet. Gynecol., 182:170-175 (2000), which is hereby incorporated by reference in its entirety), the relation between the matrix biomarkers measured in the first week of life of a premature neonate, and the subsequent bill issued for the costs of the neonatal intensive care of that patient was also analyzed. Again, a remarkable, highly significant relation was noted. Table 1 and Table 2 summarize the findings for the individual biomarkers, and a set of ratios formed mathematically from the primary data.

TABLE 1
First Week-Of-Life (fw) Matrix Biomarkers And Length-Of-Stay
In Neonatal Intensive Care

| | | Spearman | |
|---|---|---|---|
| | | correlation with | |
| | Parameter | length-of-stay | P value |
| Matrix component | fwC-IV | −0.3 | 0.03 |
| | fwP1 | 0.5 | 0.006 |
| Collagen vs. non-collagen | fwC-IV/fwP1 | −0.7 | 0.000 1 |
| | fwPICP/fwP1 | −0.72 | 0.0001 |
| | fwC-IV + fwPICP/fwP1 | −0.83 | 0.0001 |
| Collagen vs. collagen | fwC-IV/fwPINP | −0.4 | 0.03 |
| | fwC-IV/fwPIIINP | −0.47 | 0.0027 |
| | fwC-IV/fwPINP + fwPIIINP | −0.45 | 0.15 |

TABLE 2
First Week-Of-Life (fw) Matrix Biomarkers
And Costs Of Neonatal Intensive Care

| | | Spearman | |
|---|---|---|---|
| | | correlation with | |
| | Parameter | total NICU bill | P value |
| Matrix component | fwPIIINP | 0.45 | 0.01 |
| | fwPINP | 0.5 | 0.03 |
| | fwP1 | 0.7 | 0.0007 |
| Collagen vs. non-collagen | fwC-IV/fwP1 | −0.46 | 0.03 |
| | fwC-IV + fwPICP/fwP1 | −0.57 | 0.01 |
| Collagen vs. collagen | fwC-IV/fwPINP | −0.5 | 0.03 |
| | fwC-IV/fwPIIINP | −0.5 | 0.009 |
| | fwC-IV/fwPINP + fwPIIINP | −0.53 | 0.01 |

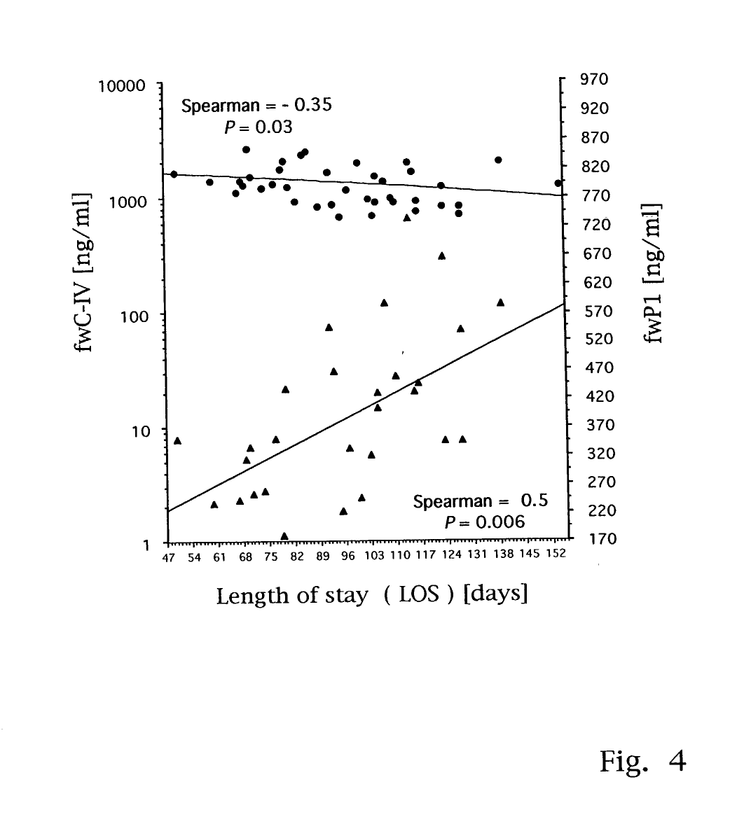

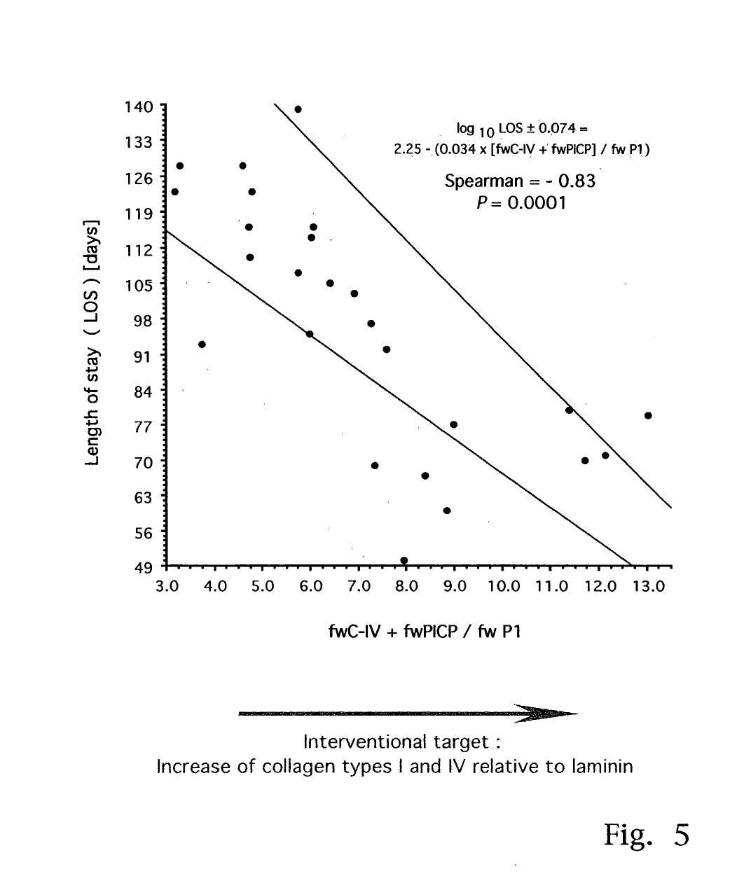

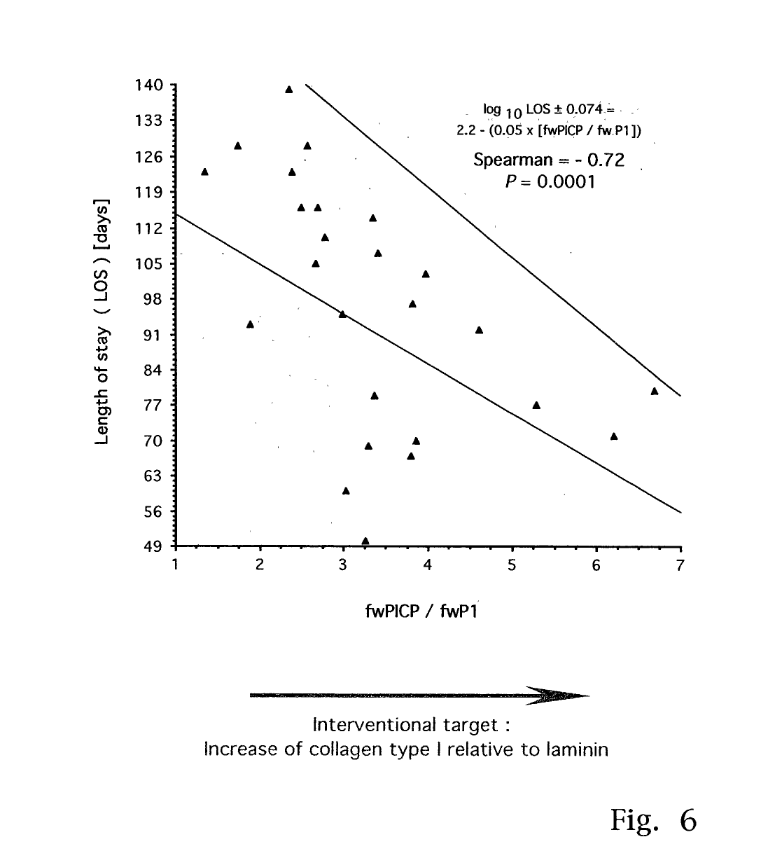

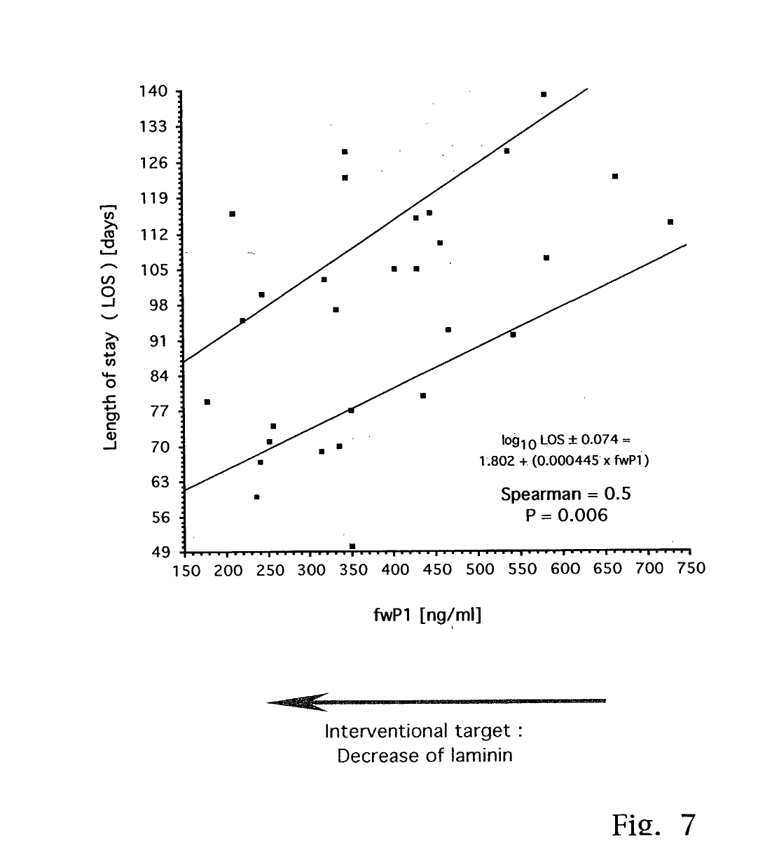

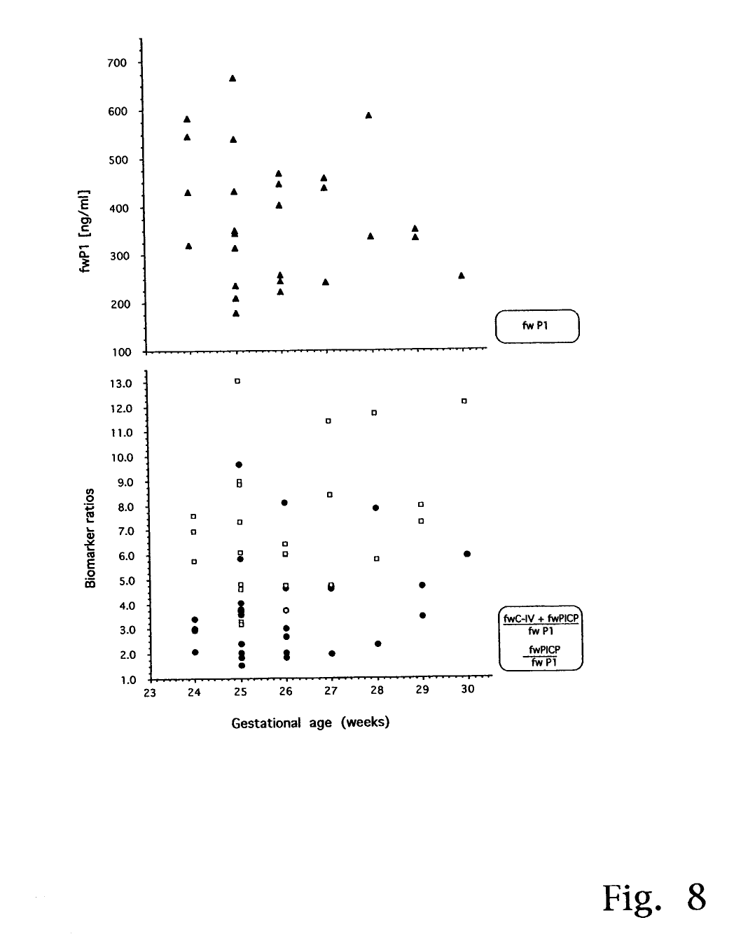

With regard to length-of-stay, the two major components that assemble the basement membrane, collagen type IV and laminin, behaved in an opposing manner, as shown in FIG. 4, for all individuals of the cohort: The higher fwC-IV, the shorter the stay; the higher fwP1, the longer the stay. Thus, the first-week parameters can be used individually or as mathematical ratios to assess the potential for a prolonged stay and the anticipated need for medical resources. Examples for the mathematical prediction of length-of-stay on the basis of fw matrix biomarker measurements are given in FIGS. 5, 6, and 7. These figures teach what medical intervention likely decreases the length-of-stay: an increase in the amount of collagen relative to the amount of laminin. As shown in FIG. 8, the biomarkers and the biomarker ratios presented in FIGS. 5, 6, and 7 do not relate to gestational age.

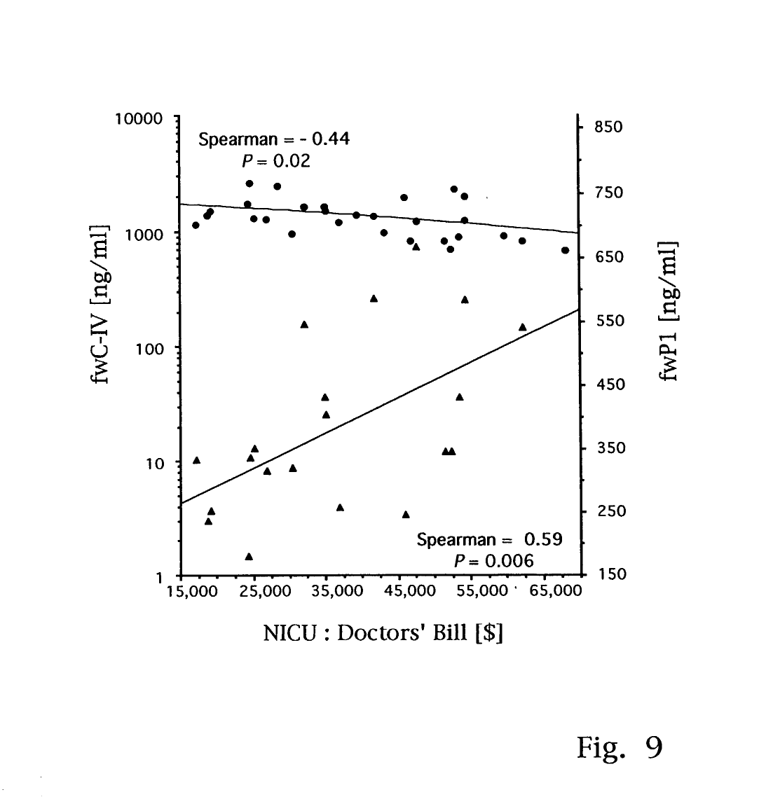

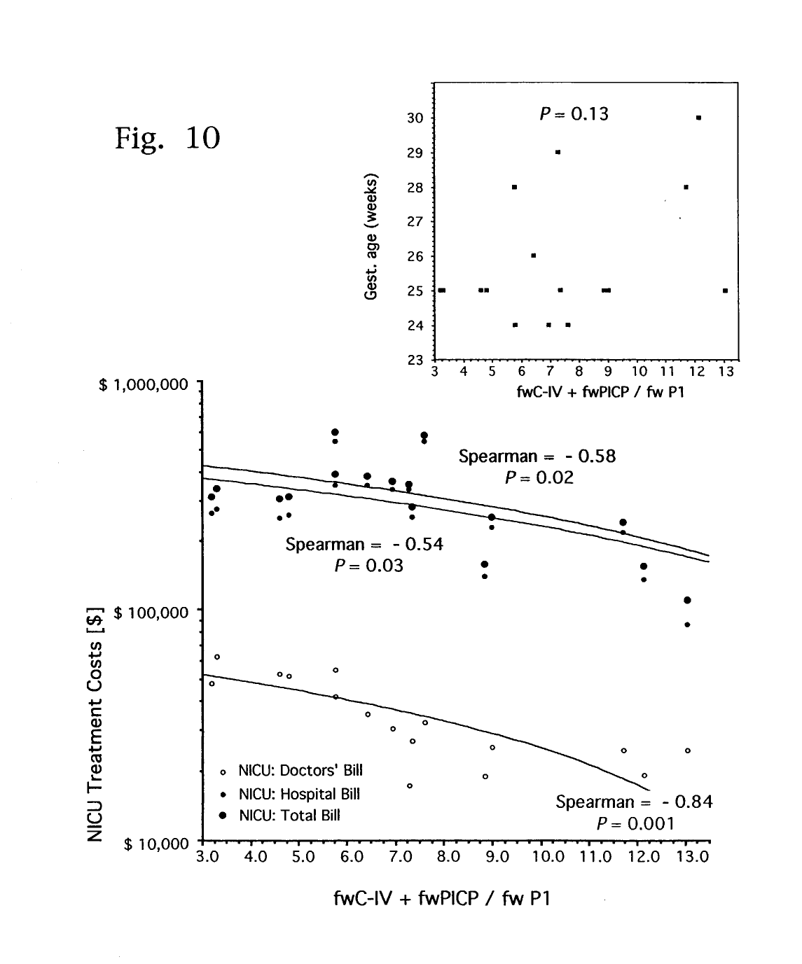

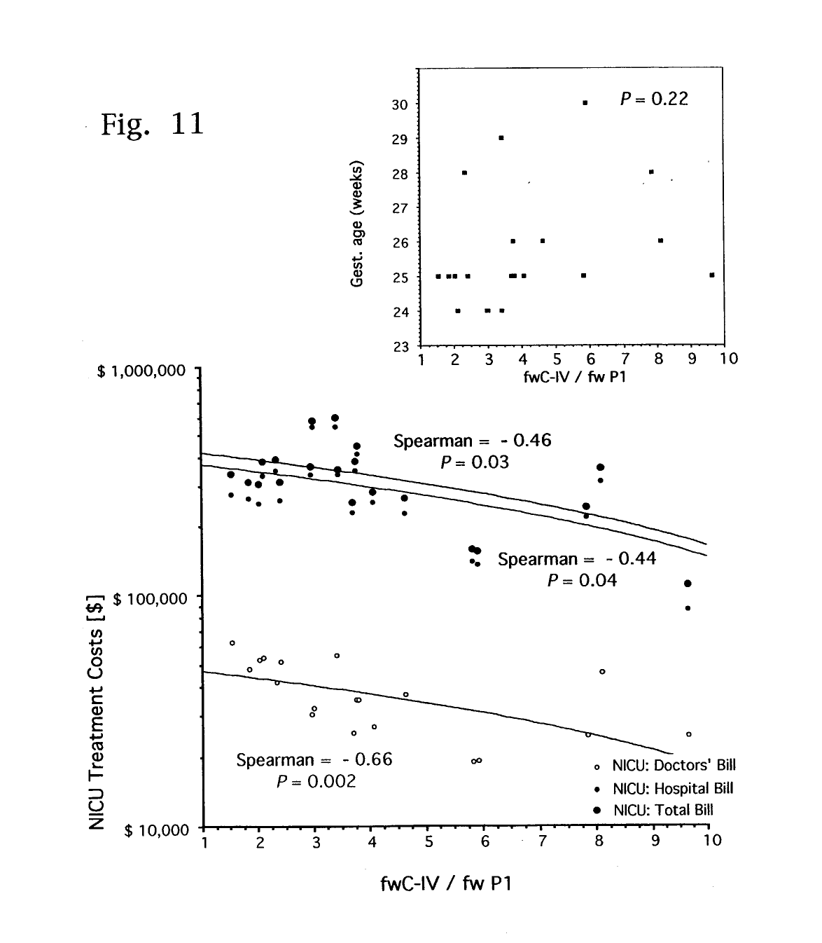

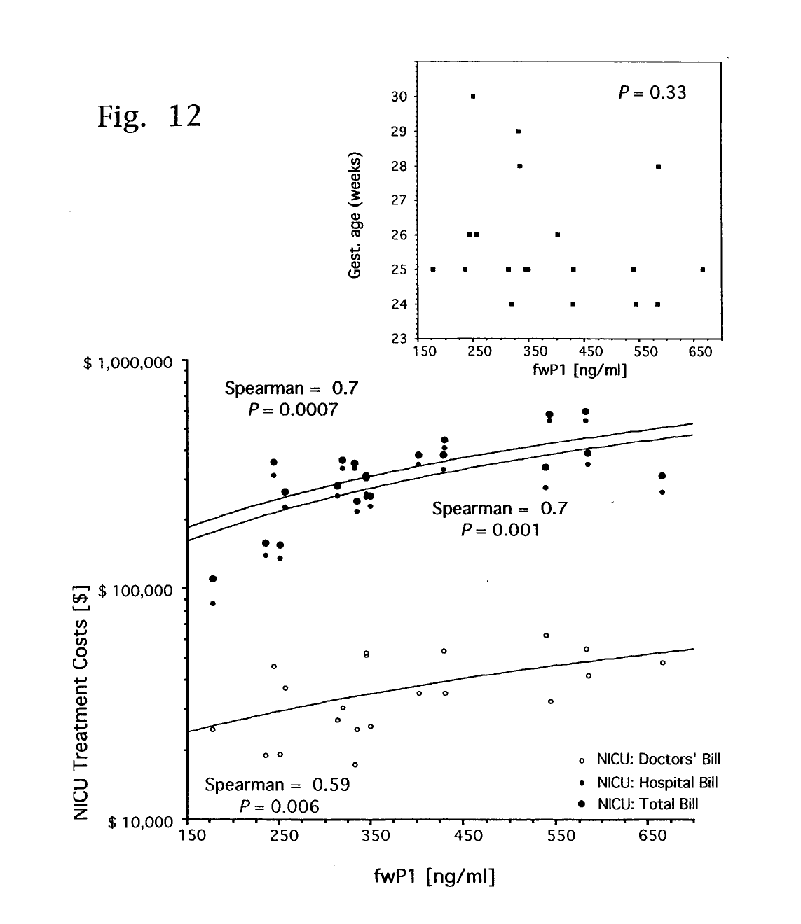

With regard to costs of neonatal intensive care, the relation between the fw matrix biomarkers and the bills issued for the direct service of physicians (“Doctors' Bill”), the charges of the hospital for supportive services rendered (“Hospital bill”), and the sum of both (“Total Bill”) was analyzed. Again, the two major components that assemble the basement membrane, collagen type IV and laminin, behaved in an opposing manner, as shown in FIG. 9, for the Doctors' Bill of all patents studied: the higher fwC-IV, the lower the physician costs; the higher fwP1, the higher the physician costs. Thus, the first-week parameters can be used individually or as mathematical ratios to assess the prospective costs incurred. Examples for the mathematical prediction of each of the three cost parameters on the basis of fw matrix biomarker measurements are given in FIGS. 10, 11 and 12. The inset in each figure shows that gestational age itself does not relate to any of the shown fw biomarker parameters that is significant for assessing the costs incurred subsequently. These figures teach what medical intervention likely decreases each of the three cost categories for NICU care of premature neonates: an increase in the amount of collagen relative to the amount of laminin.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of enhancing organ maturity of premature human neonates comprising:
administering to a premature human neonate a growth factor under conditions effective to enhance organ maturation.

2. A method according to claim 1, wherein the growth factor is selected from the group consisting of endocrine growth factors, embodied by those encoded on human chromosome 17, on chromosome 12, on chromosome 11, and on chromosome 6.

3. A method according to claim 2, wherein the growth factor is encoded on human chromosome 17 and is selected from the group consisting of hGH-N, hGH-V, chorionic somatomammotropin-A, and chorionic somatomammotropin-B.

4. A method according to claim 2, wherein the growth factor is encoded on human chromosome 12 and is IGF-1.

5. A method according to claim 2, wherein the growth factor is encoded on human chromosome 11 and is IGF-2.

6. A method according to claim 2, wherein the growth factor is encoded on human chromosome 6 and is prolactin.

7. A method according to claim 1, wherein the growth factor is selected from the group of matrix growth factors, embodied by the laminins that are encoded on various human chromosomes.

8. A method according to claim 1, wherein the conditions effective to enhance organ maturation are effective to increase basement membranes in organs of premature human neonates.

9. A method according to claim 8, wherein the conditions effective to enhance organ maturation are effective to increase collagen type IV formation in organs of premature human neonates.

10. A method according to claim 1, wherein the organ is selected from the group consisting of lung, kidney, gastrointestinal tract, heart and skeletal muscle, and blood vessels.

11. A method according to claim 10, wherein the organ is lung.

12. A method according to claim 11, wherein said administering is carried out by inhalation.

13. A method according to claim 1, wherein said administering is carried out by inhalation, orally, subcutaneously, intravenously, intramuscularly, intraperitoneally, or by application to mucous membranes.

14. A method of predicting a premature human neonate's length of stay in a neonatal intensive care unit as well as the anticipated medical costs incurred during said stay, comprising:
providing a sample from the premature human neonate;
determining biomarkers derived from extracellular matrix in the sample; and
comparing biomarker levels or ratios thereof in the sample to a standard to ascertain the premature human neonate's length of stay in a neonatal intensive care unit as well as the anticipated medical costs incurred during said stay.

15. A method according to claim 14, wherein the sample is obtained as plasma, serum, or urine.

16. A method according to claim 14, wherein the biomarkers derived from the extracellular matrix originate from collagenous proteins.

17. A method according to claim 16, wherein the biomarkers derived from the extracellular matrix originate from collagen type IV in organs of premature human neonates.

18. A method according to claim 14, wherein the biomarkers derived from the extracellular matrix originate from non-collagenous proteins or glycosaminoglycans.

19. A method according to claim 14, wherein the biomarkers derived from the extracellular matrix originate from laminin in organs of premature human neonates.

20. A method of enhancing organ maturity of premature human neonates comprising:
administering to a premature human neonate a gene encoding a growth factor under conditions effective to enhance organ maturation.

21. A method according to claim 20, wherein the gene encoding the growth factor is selected from the group consisting of genes encoding endocrine growth factors, embodied by those encoded on human chromosome 17, on chromosome 12, on chromosome 11, and on chromosome 6.

22. A method according to claim 21, wherein the growth factor is encoded on human chromosome 17 and is selected from the group consisting of hGN-N, hGH-V, chorionic somatomammotropin-A, and chorionic somatomammotropin-B.

23. A method according to claim 21, wherein the growth factor is encoded on human chromosome 12 and is IGF-1.

24. A method according to claim 21, wherein the growth factor is encoded on human chromosome 11 and is IGF-2.

25. A method according to claim 21, wherein the growth factor is encoded on human chromosome 6 and is prolactin.

26. A method according to claim 20, wherein the gene encoding the growth factor encodes a matrix growth factor, embodied by the laminins that are located on various human chromosomes.

27. A method according to claim 20, wherein the conditions effective to enhance organ maturation are effective to increase basement membranes in organs of premature human neonates.

28. A method according to claim 27, wherein the conditions effective to enhance organ maturation are effective to increase collagen type IV formation in organs of premature human neonates.

29. A method according to claim 20, wherein the organ is selected from the group consisting of lung, kidney, gastrointestinal tract, heart and skeletal muscle, and blood vessels.

30. A method according to claim 20, wherein the organ is lung.

31. A method according to claim 30, wherein said administering is carried out by inhalation.

32. A method according to claim 20, wherein said administering is carried out by inhalation, orally, subcutaneously, intravenously, intramuscularly, intraperitoneally, or by application to mucous membranes.

33. A method of enhancing organ maturity of premature human neonates comprising:
administering to a premature human neonate an agent that increases growth factor formation under conditions effective to enhance organ maturation.

34. A method according to claim 33, wherein the agent increases formation of growth factors embodied by those encoded on human chromosome 17, on chromosome 12, on chromosome 11, and on chromosome 6.

35. A method according to claim 34, wherein the growth factor is encoded on human chromosome 17 and is selected from the group consisting of hGH-N, hGH-V, chorionic somatomammotropin-A, and chorionic somatomammotropin-B.

36. A method according to claim 34, wherein the growth factor is encoded on human chromosome 12 and IGF-1.

37. A method according to claim 34, wherein the growth factor is encoded on human chromosome 11 and is IGF-2.

38. A method according to claim 34, wherein the growth factor is encoded on human chromosome 6 and is prolactin.

39. A method according to claim 33, wherein the agent increases formation of matrix growth factors embodied by laminins that are encoded on various chromosomes.

40. A method of increasing collagen type IV formation in a living system comprising:
administering a growth factor, a gene encoding a growth factor, or an agent that increases growth factor formation to the living system under conditions effective to increase collagen type IV formation.

41. A method according to claim 40, wherein a growth factor is administered.

42. A method according to claim 41, wherein the growth factor is selected from the group consisting of endocrine growth factors, embodied by those encoded on human chromosome 17, on chromosome 12, on chromosome 11, and on chromosome 6.

43. A method according to claim 42, wherein the growth factor is encoded on human chromosome 17 and is selected from the group consisting of hGH-N, hGH-V, chorionic somatomammotropin-A, and chorionic somatomammotropin-B.

44. A method according to claim 42, wherein the growth factor is encoded on human chromosome 12 and is IGF-1.

45. A method according to claim 42, wherein the growth factor is encoded on human chromosome 11 and is IGF-2.

46. A method according to claim 42, wherein the growth factor is encoded on human chromosome 6 and is prolactin.

47. A method according to claim 41, wherein the growth factor is selected from the group of matrix growth factors, embodied by the laminins that are encoded on various human chromosomes.

48. A method according to claim 40, wherein a gene encoding a growth factor is administered.

49. A method according to claim 48, wherein the gene encoding the growth factor is selected from the group consisting of genes encoding endocrine growth factors, embodied by those encoded on human chromosome 17, on chromosome 12, on chromosome 11, and on chromosome 6.

50. A method according to claim 49, wherein the growth factor is encoded on human chromosome 17 and is selected from the group consisting of hGH-N, hGH-V, chorionic somatomammotropin-A, and chorionic somatomammotropin-B.

51. A method according to claim 49, wherein the growth factor is encoded on human chromosome 12 and is IGF-1.

52. A method according to claim 49, wherein the growth factor is encoded on human chromosome 11 and is IGF-2.

53. A method according to claim 49, wherein the growth factor is encoded on human chromosome 6 and is prolactin.

54. A method according to claim 48, wherein the growth factor is selected from the group of matrix growth factors, embodied by the laminins that are encoded on various human chromosomes.

55. A method according to claim 40, wherein an agent that increases growth factor formation is administered.
